(19) United States
(12) Patent Application Publication
(10) Pub. No.: US 2024/0044858 A1
(43) Pub. Date: Feb. 8, 2024

(54) ELECTRO-OPTICAL SENSING OF A TARGET GAS COMPOSITION

(71) Applicant: Analog Devices, Inc., Wilmington, MA (US)

(72) Inventors: Max Marcel Shulaker, Cambridge, MA (US); Samuel H. Fuller, Wellesley, MA (US)

(21) Appl. No.: 18/229,423

(22) Filed: Aug. 2, 2023

Related U.S. Application Data
(60) Provisional application No. 63/370,223, filed on Aug. 2, 2022.

(51) Int. Cl. G01N 33/00; G01N 21/65

(52) U.S. Cl. CPC G01N 33/0062; G01N 21/65

(57) ABSTRACT

A target gas composition, such as within ambient gas in an environment, can be detected using a gas chemical detector. A functionalized region of the gas chemical detector can be exposed to the ambient gas. The functionalized region can include an optical property indicative of the target gas composition. An optical response signal can be generated based on the optical property and indicative of the target gas composition within the ambient gas in the environment using the functionalized region. An electrical property can be electrochemically transduced, the electrical property indicative of the target gas composition into an electrical response signal. Both the electrical and optical properties can be used together to determine a presence or other characteristic of a target gas in an ambient environment, such as can be produced by a bacteria or infectious agent of interest.

### CLAIM OF PRIORITY

This application claims priority to U.S. Provisional Application Ser. No. 63/370,223, filed Aug. 2, 2022, which is hereby incorporated by reference herein in its entirety.

### BACKGROUND

Certain techniques can assist in rapid identification of an infectious agent in a biological specimen, such as for use in identifying infections such as pneumonia, sepsis, meningitis, Lyme disease, and hepatitis. For example, several diagnostic tests, especially those used to detect infectious agents, do not provide a result that is available within a short period of time, such as on the order of two hours or less. For example, a polymerase chain reaction (PCR) procedure is a nucleic acid amplification technique that amplifies and identifies nucleic acid sequences in a sample of the specimen that are unique to a particular organism or to a strain of a given organism. While a PCR technique can help identify the presence of an organism in a specimen in very low numbers, the technique presents a challenge in that it can include a complicated and time-consuming procedure, which requires expensive and sophisticated equipment.

For example, random mutagenesis-PCR (RM-PCR) can be used such as with matrix-assisted laser desorption/ionization (MALDI) mass spectroscopy to help identify an infectious agent in a biological specimen. The agent is incubated with a sample containing RM-PCR reagents, such as the PCR primers and DNA polymerase, to generate complementary DNA (cDNA) copies of the genomes of a plurality of variants of the agent. The PCR products generated can then be analyzed using conventional PCR and DNA sequencing methods. A challenge associated with such a method is the relative efficiency of the mutagenesis process (e.g., the yield of mutationally-induced variants). While random mutagenesis is generally faster, RM-PCR produces relatively few mutations per round of selection, resulting in a slow testing process.

Alternatively, chromatography and mass spectroscopy can be used to detect certain gas compositions associated with a target organism. For example, a Gas Chromatography—Mass Spectroscopy (GC-MS) or a Gas Chromatography—Flame Ionization Detection (GC-FID) machine can be used with microfluidic systems to screen a specimen containing a target organism for a targeted gas constituent, such as one that could be found to be present only in the presence of a target organism. A GC-MS machine can be used to detect certain gases of diagnostic interest, e.g., carbon dioxide, oxygen, and nitrogen. However, such a technique can have a relatively poor dynamic range, slow testing procedure, and an inherently slow detection rate. For example, a GC-MS machine can be used to detect certain gas constituents, such as ethanol, in the blood of a patient who might be suspected of being under the influence of an alcohol containing substance, such as might be found in the test of a driver suspected of being under the influence of alcohol. But for a specimen for which there is not a suspicion of a particular infectious agent, such as might be found in the test of a patient with pneumonia or sepsis, the detection range of such machines can be inadequate.

### SUMMARY

This document describes approaches to detection of a target gas composition within ambient gas in an environment, such as using a gas chemical detector including a functionalized region. Such a functionalized region can include an optical property indicative of the target gas composition. An electrical property of the target gas can be detected along with the optical property of the functionalized region. For example, the electrical property can be different than the optical property and at least one of the electrical and optical properties can be associated with a change of the functionalized region to distinguish the target gas composition from other ambient gases.

The functionalized region of the electrochemical sensor can include one or more functionalization materials such as a coating, film, membrane, or a combination thereof. The functionalization material can be susceptible to electrically or optically distinguishable characteristics in a presence of the target gas composition. For example, the functionalized region can include carbon nanotubes or other similar semiconductors or conductors. The carbon nanotubes, or other similar component, can be functionalized with the one or more functionalization materials (e.g., oligonucleotides, metal coordination complexes, porphyrins, self-assembled monolayers (SAMs), polymers, pyrrole derivatives, phthalocyanines, nanomaterial decorations, dendrimers, fluorophores, nanoparticles, nanoparticles decorated with one or more moieties, or the like). The one or more functionalization materials can alter one or more electronic, thermal, or optical properties detected at the functionalized region in the presence of the target gas composition. Measurement of various optical and electrical parameters at a plurality of functionalization regions can contribute to a qualitative or quantitative assessment of the gas concentration.

While it may be desirable to detect the target gas composition from a gas source that has a relatively higher concentration (e.g., 1-10%) of the target gas composition, functionalized carbon nanotubes can enable the measurement of the target gas composition from a gas source that has a relatively lower concentration (e.g., <1%) of the target gas composition. This capability can be advantageous for use in a variety of applications, including but not limited to, detecting volatile organic compounds (VOCs), and measuring the concentration of a target gas in a breath sample.

For example, electromagnetic energy such as visible light from a light source can be received at the functionalized region. An optical response signal can be generated using the functionalized region, such as based on the optical property and indicative of the target gas composition within the ambient gas in the environment. An electrical property can be electrochemically transduced, and the electrical property can be indicative of the target gas composition into an electrical response signal. Using both the optical response signal and the electrical response signal, a presence or other characteristic of the target gas composition within the ambient gas in the environment can be determined.

The functionalized region can be imaged such as to produce an image-readable characteristic indicative of the target gas composition within the ambient gas in the environment using the functionalized region. For example, image-processing an image of the functionalized region can be performed such as to detect the image-readable characteristic. Here, the image-readable characteristic can be used such as to help generate at least a portion of the optical response signal indicative of the presence or other characteristic of the target gas composition within the ambient gas in the environment.

Also, the functionalized region can be illuminated with the electromagnetic energy such as via a broadband or a tunable wavelength light source. Thus, at least a portion of the optical response signal based on the optical property and indicative of the target gas composition within the ambient gas in the environment can be generated in response to the illumination using the functionalized region.

In an example, the generating at least a portion of the optical response signal can include generating spectral response data including at least one of absorption, reflection, fluorescence, elastic scattering, or inelastic (Raman) scattering of the functionalized region, in response to the illuminating, indicative of the presence or other characteristic of the target gas composition within the ambient gas in the environment at the functionalized region.

In an example, the illumination can be variable between a plurality of different illuminations. Here, at least a portion of the optical response signal indicative of the target gas composition within the ambient gas in the environment can be generated in response to the different illuminations using the functionalized region. The optical response signal at the different illuminations can be processed such as to determine a presence or other characteristic of the target gas composition within the ambient gas in the environment.

In an example, the electrical property can be electrochemically transduced into the electrical response signal using the same or a different functionalized region of the gas chemical detector. The optical response signal indicative of the target gas composition within the ambient gas in the environment can be generated using the functionalized region, which can include using, e.g., a Field Effect Transistor (FET) associated with the functionalized region.

In an example, the FET can be electrically biased at a specified bias level such as to help to determine a presence or other characteristic of the target gas composition within the ambient gas in the environment. Also, an electrical property of the FET can change in response to exposure of the functionalized region to ambient gas including the target gas composition. For example, the changed electrical property of the FET can include at least one of an effective gate voltage of the FET, an effective channel resistance of the FET, an effective channel conductance of the FET, an effective transconductance of the FET, or at least one of a gate-body, gate-drain, or gate-source interface property of the FET.

Processing to determine a presence or other characteristic of the target gas composition within the ambient gas in the environment can include comparing or analyzing spectral response data from the optical response signal using at least one of a library template or trained model.

Gas chemical detector devices described herein can include a sensor including the functionalized region. The sensor can include at least one of an electrochemical transducer, a photodetector, or a colorimetric imaging sensor. In an example, the sensor can be located facing an opposing illuminator, and arranged to permit the ambient gas from the environment to enter a space between the sensor and the illuminator.

Gas chemical detector devices described herein can also include at least one carbon nanotube field-effect transistor (CNFET), such as an array of CNFETs. For example, individual ones of the CNFETs in the array of CNFETs can correspond to different functionalized regions corresponding to different target gas compositions.

Each of the non-limiting examples described herein can stand on its own, or can be combined in various permutations or combinations with one or more of the other examples.

This Summary is intended to provide an overview of the subject matter of the present patent application. It is not intended to provide an exclusive or exhaustive explanation of the invention. The detailed description is included to provide further information.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1A depict an example of a gas chemical detector device including an enclosure.

FIG. 1B depicts an electrochemical transducer.

FIG. 1C depicts an electrochemical transducer including a plurality of sensors respectively including a functionalized region.

FIG. 2A depicts an example of a sensor included in an electrochemical transducer.

FIG. 2B depicts an example of an electrochemical transducer including an array of sensors.

FIG. 2C is a detailed view depicting an example of a sensor included in an electrochemical transducer.

FIG. 3A depicts a perspective view of an example of a portion of an example of a sensing unit.

FIG. 3B depicts a perspective view of an example of a portion of an example of a sensing unit.

FIG. 4A depicts a perspective view of an example of a portion of an example of a sensing unit.

FIG. 4B depicts a perspective view of an example of a portion of an example of a sensing unit.

FIG. 5 depicts a model chart including examples of profiles associated with different species of infectious agents.

FIG. 6 is a block diagram of an example of a gas chemical detector device.

FIG. 7 is a block diagram of another example of a gas chemical detector device.

FIG. 8 is a block diagram of another example of a gas chemical detector device.

FIG. 9 is a flowchart that describes a method for detecting a target gas composition.

FIG. 10 is a block diagram illustrating components of a machine.

### DETAILED DESCRIPTION

This document describes generally, among other things, devices, and methods for detecting a target gas composition. More particularly, this document describes detecting a target gas composition within ambient gas in an environment using a gas chemical detector with a functionalization material.

An approach to identification of an infectious agent within a biological specimen can include analysis of volatile organic compounds (VOCs) emitted from the biological specimen. Some infectious bacteria can produce a distinguishable characteristic VOC composition that can, using a gas detection technique, can indicate a presence or other characteristic of the bacteria. One technique for detecting the presence of a target gas can involve using a gas chromatograph (GC) to analyze a specimen. For example, a biological specimen sample can be subjected to a chemical or physical separation that extracts or purifies the VOCs from the sample. Thereafter, the GC measures, identifies, and quantifies the VOCs by providing a gas chromatographic profile for the sample. The chromatographic profile provides a pattern of peaks that indicate the relative abundances of different compounds (i.e., a peak or peaks that indicate a certain concentration of a particular compound) as a function of time (i.e., as a function of GC column). As a result of the analysis of the chromatographic profile, a presence, concentration, or other characteristic of a target gas in the specimen can be determined.

A challenge with such GC techniques is that they can require extensive sample preparation or extensive gas chromatographic analysis. More specifically, because of the need to obtain an accurate sample and to run the GC to completion, these techniques can be relatively lengthy. Moreover, these techniques may not be adequately sensitive for certain applications, such as being unable to detect small quantities of target gas in the specimen. Thus, GC methods may not be suited for use in detecting target gases on short time scales. Another challenge with the above-described GC techniques is that they may employ complex methods for extracting or purifying VOCs from a specimen and separating the VOCs from other ambient air constituents. For example, certain GC systems can require that the sample be drawn through a sample line. This can be done via a vacuum or an aspirating pump. To ensure a consistent sample for analysis, these systems require a substantially constant flow rate. But these systems have a long dead volume (i.e., the volume of the system not in contact with the sample) that can contain some of the sample being analyzed. To increase the efficiency of the vacuum system, the flow through the line must not vary significantly. In an approach to achieve an adequately constant flow, a high mass transfer area is used, requiring relatively large volume pumping elements. This approach to GC sensing hinders the ability to fit an accurate gas-sensing system in small, or portable, field kits or in other locations in which space and weight are at a premium.

The present inventors have recognized a need for a gas sensing technique with improved sensitivity and including a smaller, such as more portable, form factor. Devices and methods for detection of a target gas composition within ambient gas in an environment are described herein. For example, a gas chemical detector can include or use a functionalized region. Such a functionalized region can include an optical property indicative of the target gas composition. An electrical property of the target gas can be detected along with (e.g., concurrent with) the optical property of the functionalized region. The electrical property can be different than the optical property. At least one of the electrical property and the optical property can be associated with a change of the functionalized region and used to distinguish the target gas composition from other ambient gases. For example, concurrently measuring both the electrical property and the optical property can help improve the ability to distinguish between different infectious agents within a biological specimen.

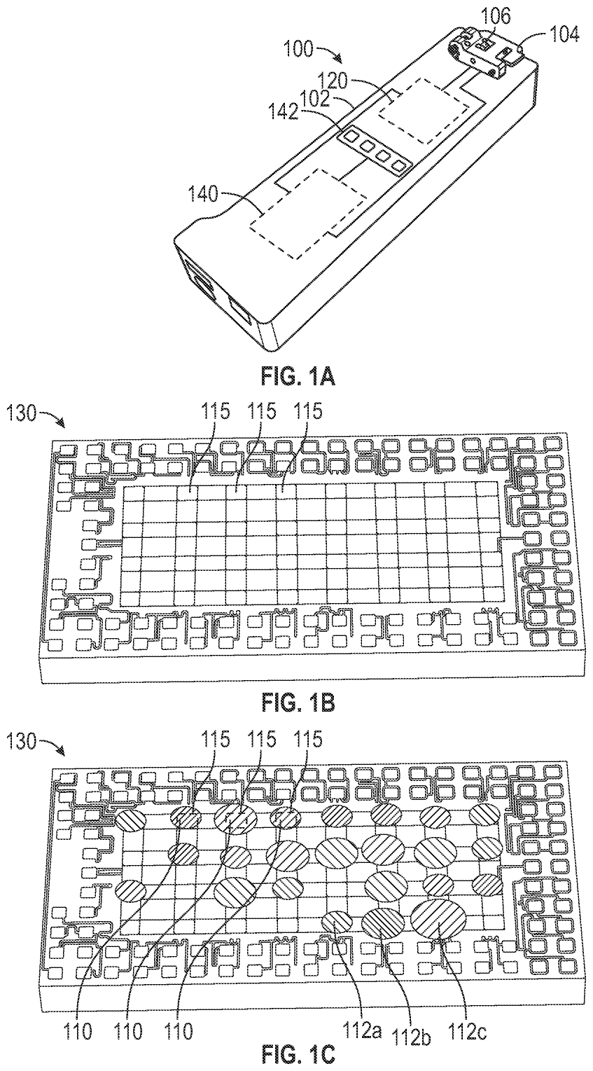

FIG. 1A, FIG. 1B, and FIG. 1C depict an example of a gas chemical detector device 100. As shown in FIG. 1A, a gas chemical detector device 100 can include an enclosure 102 for housing gas chemical detector componentry therein. The enclosure can be sized and shaped such as to be held in a human hand, e.g., for portable gas-sensing or as a part of a field kit. The gas chemical detector device 100 can also include a sensing unit 104 including at least one gas transmission opening 106 (e.g., one or more holes, channels, or pores) to allow gas transmission from the ambient environment to reach the gas chemical detector componentry.

FIG. 1B depicts an electrochemical transducer 130 including an array of sensors. The sensing unit 104 can also include or use an electrochemical transducer 130 for transducing at least one electrical property indicative of the target gas composition into an electrical response signal. For example, the electrochemical transducer 130 can include at least one sensor 115, such as an array of sensors 115. For example, an individual sensor 115 can include an electrochemical sensing element such as a Field Effect Transistor (FET), a p-n junction diode, an electrochemical cell, a conductive polymer or electrode, or other semiconductor or other device that, when exposed to a particular target gas composition, changes a signal (e.g., current, voltage, or impedance) in accordance with a presence or concentration of the target gas composition.

FIG. 1C depicts an electrochemical transducer 130 including a plurality of sensors respectively including a functionalized region. In an example, an individual sensor 115 can include or use at least one functionalized region 110. The functionalized region 110 can include a covering or coating including at least one functionalization material 112 over the electrochemical sensing element of an individual sensor 115. Also, the at least one functionalization material 112 can be otherwise present at or formed with the electrochemical sensing element of an individual sensor 115. As depicted in FIG. 1C, the electrochemical transducer 130 can include a plurality of sensors 115 respectively including different functionalization materials such as functionalization materials 112a, 112b, and 112c. In an example, the different functionalization materials can include an oligonucleotide, a metal coordination complex, a porphyrin, a self-assembled monolayer (SAM), a polymer, a pyrrole derivative, a phthalocyanine, or a nanomaterial decoration.

Returning to FIG. 1A, in an example the gas chemical detector device 100 can also include detector circuitry 120 arranged such as to generate an optical response signal indicative of the target gas composition. For example, the detector circuitry 120 can use an optical characteristic of an individual functionalized region 110 such as absorption, reflection, fluorescence, elastic scattering, or inelastic (Raman) scattering, of at least one functionalization material 112 included in the functionalized region 110.

The gas chemical detector device 100 can also include signal processing circuitry 140, using both the optical response signal and the electrical response signal to determine a presence or other characteristic of the target gas composition within the ambient gas in the environment. In an example, the signal processing circuitry 140 can be communicatively coupled with a user input 142 such as a keypad, a touchscreen, or a similar device that a user can use to communicate a user input to the signal processing circuitry.

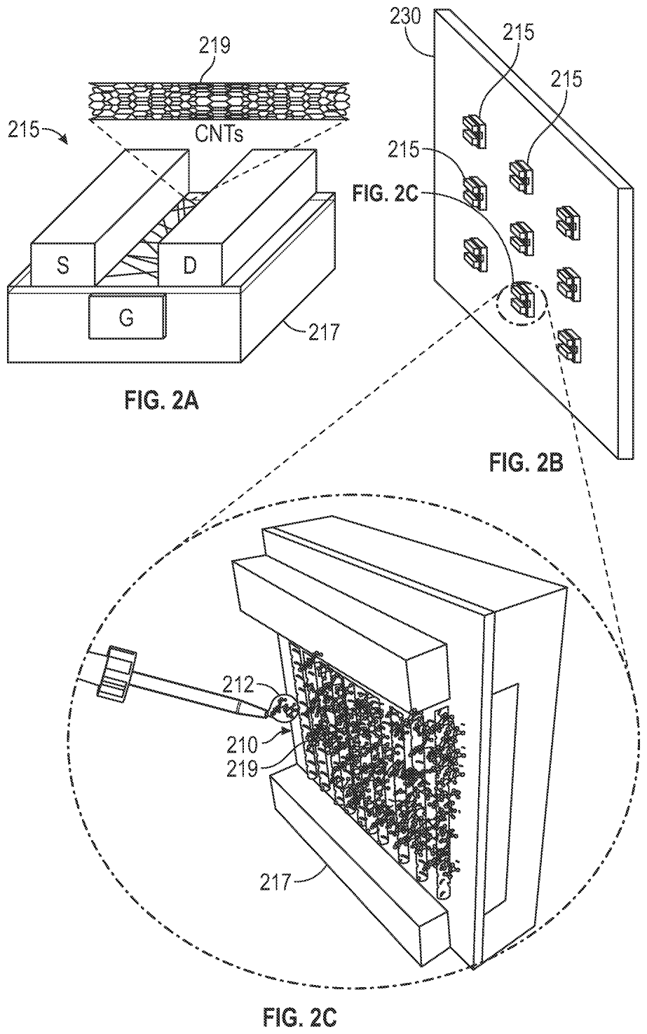

FIG. 2A depicts an example of a sensor included in an electrochemical transducer. In an example, a sensor 215 of an electrochemical transducer 230 (as depicted in FIG. 2B) can include or use at least one carbon nanotube field-effect transistor (CNFET) 217. As depicted, a CNFET can include source (S), drain (D), and gate (G) regions. A CNFET is a type of transistor that uses the conduction of various carbon nanotubes rather than a bulk semiconductor channel region in a metal field-oxide semiconductor field-effect transistor (MOSFET). A CNFET 217 can include a channel that is formed using one or more carbon nanotubes (CNT) 219 and a gate G that is positioned above the channel, at an interface between the channel and the gate. The CNTs 219 can provide a controlled-conductivity channel between the drain and source, such as by applying a bias to the gate G. The functionalized material can modify the effective bias at the gate G in response to the presence or concentration of one or more target constituent analytes in the ambient gas in the environment. By controlling the conductivity of the channel carbon nanotubes using a gate, the electrochemical transducer can generate at least a portion of an electrical response signal for determining a presence or other characteristic of the target gas.

FIG. 2B and FIG. 2C depict an example of an electrochemical transducer 230 including an array of sensors. For example, an array of sensors 215 of an electrochemical transducer 230 can each include respective CNFETs 217. In the detailed view as shown in FIG. 2C, at least one functionalization material 212 can be applied to an individual sensor 215 such as at a CNFET 217 of the sensor 215. Application of the functionalization material 212 can include, e.g., physical evaporation, chemical vapor deposition, photolithography, spin-coating, or dip-coating of the functionalization material 212 at a functionalized region 210 the sensor 215. For example, a monolayer, bilayer, or multi-layer of a functionalization material 212 can be applied to the CNTs 219 of an individual CNFET 217.

As described further below, certain ones of the functionalization materials 212 can include optically detectable characteristics to help detect the target gas composition. For example, the functionalization material 212 may be optically detectable, or luminescent, such that it emits light upon exposure to the particular target gas composition. For example, the functionalization material 212 can include colorimetric indicator molecules that can change color or emit fluorescent light when exposed to a particular target gas composition.

The array of sensors 215 can be functionalized with a plurality of different functionalization materials 212. For example, the array of sensors 215 can include at least one functionalization material 212 including an optically detectable characteristic to help detect the target gas composition and at least one electrically detectable characteristic to help detect the same target gas composition. In another example, the array of sensors 215 can be functionalized with a plurality of functionalization materials 212 including optically or electrically detectable characteristics to help detect at least two different target gas compositions. Here, the array of sensors 215 can concurrently and individually screen for a presence or other characteristic of multiple target gases or multiple target gas compositions.

Functionalization materials 212 for application at an individual functionalization region 210 of the electrochemical transducer 230 can include, e.g., oligonucleotides, metal coordination complexes, porphyrins, self-assembled monolayers (SAMs), polymers, pyrrole derivatives, phthalocyanines, nanomaterial decorations, biotin-avidin linkages, peptides, antibodies, enzymes, or one or more combinations thereof. Also, as described further below, certain functionalization materials 212 can be included in the electrochemical sensor, such as at the functionalization region 210, for optical signal transducing and digitization before and after exposure to a target gas. Such functionalization materials can include, e.g., antibodies, polymer probes, aptamers, micro-particles with molecular targets, metal particles, fluorescent materials, silica microspheres, silica/polymer hybrid microspheres, nanocomposites with magnetic, noble, or semiconductor nanoparticles, or one or more combinations thereof.

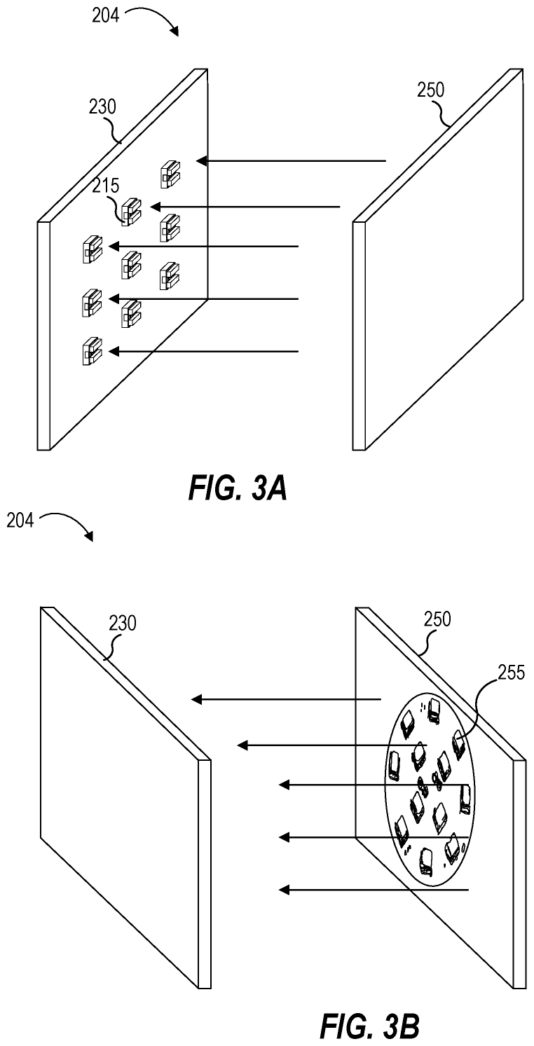

FIG. 3A and FIG. 3B depict perspective views of an example of a portion of an example of a sensing unit. A sensing unit 204 can include or use the electrochemical transducer 230 including at least one sensor 215. The at least one sensor 215 (such as an array of sensors 215, as shown) can be located facing an opposing illuminator 250 and arranged to permit the ambient gas from the environment to enter a space between the sensor 215 and the illuminator 250. The illuminator can include or use at least one spectrally specifiable or tunable illumination source 255. For example, the illumination source can include one or more visible or invisible light emitting diodes (LEDs), laser diodes (LDs), laser-diode arrays (LDAs), light emitting molecules (LEMs), or one or more combinations thereof.

In an example, the illumination source 255 can be located on a first integrated circuit die, and the first and second functionalized regions can be located on an opposing second integrated circuit die, facing the first integrated circuit die with a region therebetween exposed to the ambient gas from the environment to be sensed.

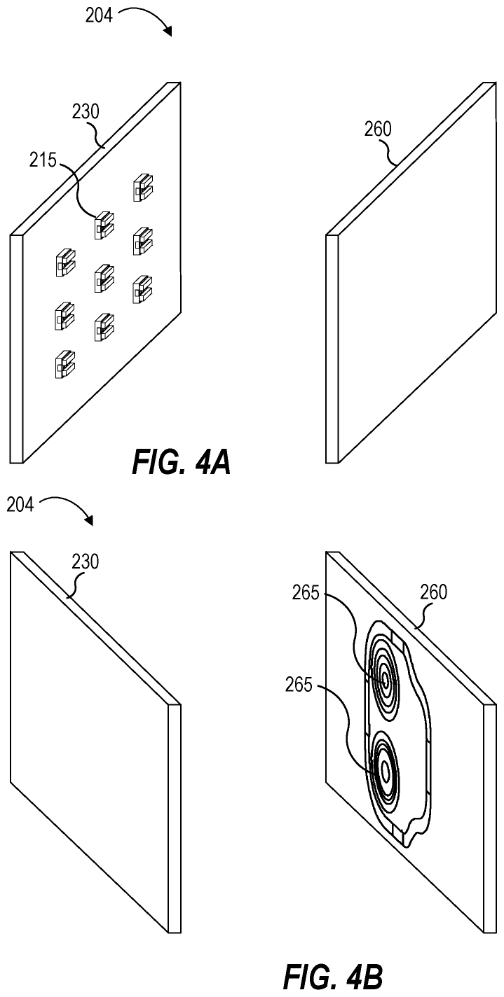

FIG. 4A and FIG. 4B depict perspective views of another example of a portion of an example of a sensing unit. A sensing unit 204 can include or use the electrochemical transducer 230 including at least one sensor 215. Here, the at least one sensor 215 (such as an array of sensors 215, as shown) can be located facing an opposing imaging unit 260 and arranged to permit the ambient gas from the environment to enter a space between the sensor 215 and the imaging unit 260. The imaging unit 260 can include or use at least one camera 265 or other similar type of imager, such as a charge-coupled device (CCD) imager or CMOS imager. For example, the camera 265 can include an RGB or other multi-spectral imaging camera (i.e., an imaging unit with at least two different wavelength-selective cameras or sensors) and can include or use a spectrograph to enable spectroscopic detection.

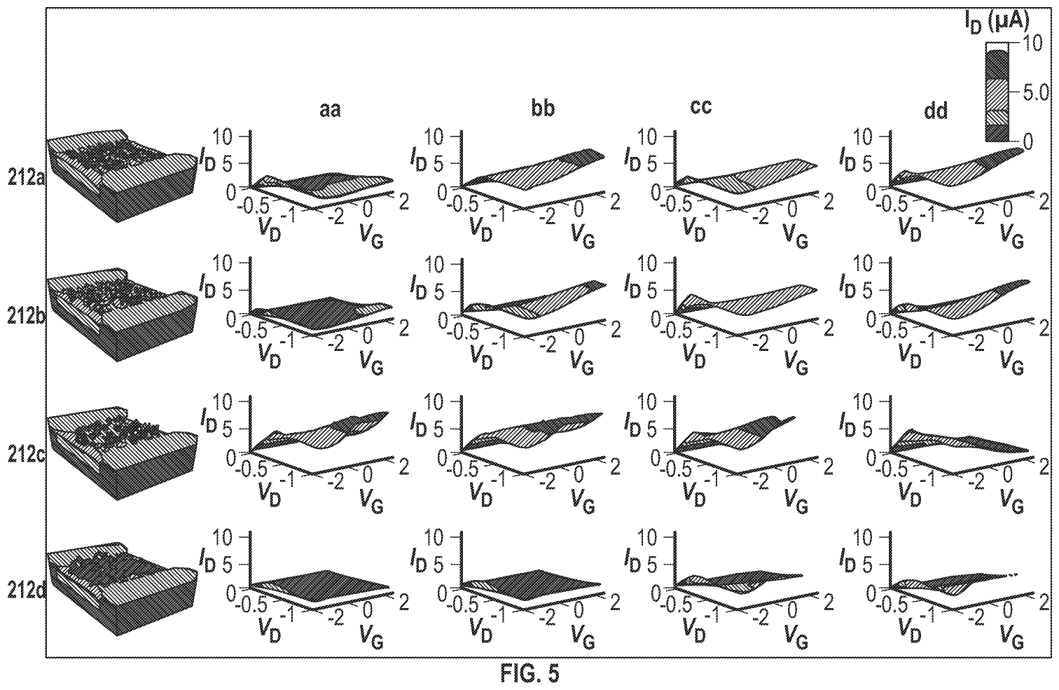

FIG. 5 depicts a model chart including exemplary profiles associated with different species of infectious agents. The exemplary model chart shown in FIG. 5 includes hypothetical data for the purpose of explanation only and is not meant to represent actual data. As shown, several different species of infectious agents aa-dd can display distinguishable gas sensor data profiles. Also, infectious agents aa-dd can display distinguishable gas sensor data profiles when exposed to several different functionalization materials 212a-212d. Thus, by analyzing received data including multiple parameters using a first functionalization material 212a, a determination can be made such as to whether any of infectious agents aa, bb, cc, or dd, are present within a given biological specimen. Such an analysis can be expanded when repeating at different functionalization materials 212b, 212c, 212d, and so on.

An individual profile for an infectious agent aa at a particular functionalization material 212a can include several parameters (such as drain current (ID), source-drain voltage (VD), and gate voltage (VG), as depicted in FIG. 5) for creating a 3-dimensional profile (i.e., an XYZ plot). Also, at least one optical parameter, such as absorption, reflection, fluorescence, elastic scattering, inelastic (Raman) scattering can be included in addition to or in alternative to any of the electrical parameters such as to create a 3-dimensional profile or other multidimensional profile. For example, VG could be replaced in the Z axis of the depicted chart with at least one of the optical parameters such as to help distinguish between different infectious agents by combined electrical and optical profiling.

In an example, received data from the sensing unit can be analyzed by comparing one or more measured electrical parameters, optical parameters, or both with at least one of a library template or a trained model in order to determine a presence or other characteristic of a particular infectious agent or other agent of interest. In an example, the library template or trained model can be created by first performing a statistical analysis on a database of at least one electrical parameter for a known infectious agent and then combining the electrical parameters with at least one of an optical parameter for the same known infectious agent. For example, a library can include or use a profile for a plurality of known infectious agents, e.g., Escherichia coli, Proteus mirabilis, Moraxella catarrhalis, Serratia marcescens, Klebsiella pneumoniae, Burkholderia epacian, Acinetobacter baumannii, Streptococcus pneumoniae, Stenotro phomonas (Xanthomonas) maltophilia, Aspergillus niger, Neisseria lactamica, Streptococcus pyogenes, Pseudomonas aeruginosa, Staphylococcus aureus, or Haemophilus influenzae. The combined electrical and optical parameter for each known infectious agent can be compared to at least one of the library template and the trained model to determine a presence of the unknown infectious agent. Also, in an example, received data from the sensing unit can be analyzed, such as using a neural network, for recognition of at least one infectious agent. For example, neural networks can be trained using data sets containing information pertaining to at least one known infectious agent and then applied to the received data to determine whether an unknown infectious agent is present. The neural network can provide statistical evidence that the unknown infectious agent is present. In some embodiments, the unknown infectious agent can be identified based on the neural network's confidence level. The neural network can be trained by providing it with training data that contains information that pertains to the presence or absence of a known infectious agent. The training data can be provided by a database having a collection of known infectious agents stored therein.

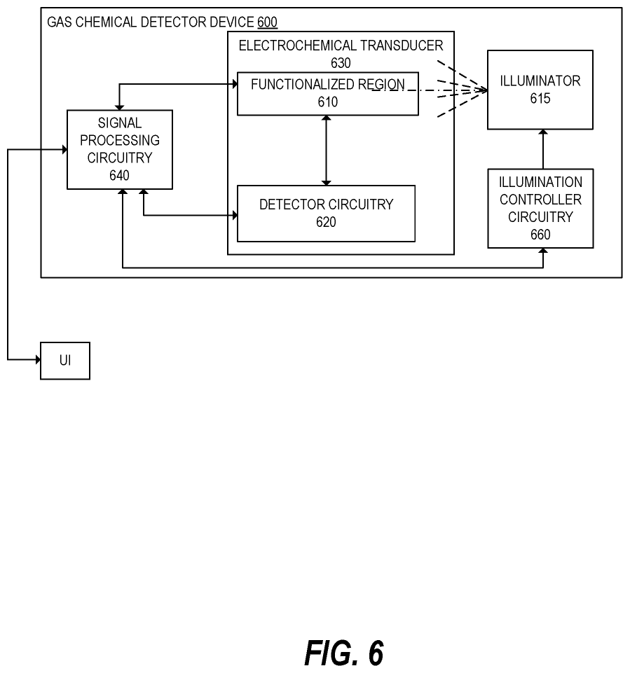

FIG. 6 is a block diagram of an example of a gas chemical detector device. A gas chemical detector device 600 can include or use an electrochemical transducer 630 including a functionalized region 610, detector circuitry 620, signal processing circuitry 640, and an illuminator 615. The illuminator 615 can include or use at least one of a broadband or tunable wavelength light source, arranged to illuminate the functionalized region 610 with electromagnetic energy while the functionalized region 610 is exposed to the ambient gas. The detector circuitry 620 can be included for generating at least a portion of the optical response signal, in response to illumination from the illuminator 615, using the functionalized region 610. The optical response signal can be indicative of the target gas composition within the ambient gas in the environment. In an example, the functionalized region 610 can be arranged to, in response to the illumination, provide optical response data.

The gas chemical detector device 600 can also include illumination controller circuitry 660. In an example, the illumination controller circuitry 660 can be configured for varying the illumination between a plurality of different illuminations, such as varying an illumination wavelength among individual ones of the plurality of different illuminations. Other variations of illuminations can include, e.g., varying an intensity, pulse duration, frequency, and/or amplitude of illumination. Other examples of illuminations include visible light, non-visible light (such as, e.g., infrared, ultraviolet, microwave, etc.), polarized light, non-polarized light, etc.

The functionalized region 610 or the detector circuitry 620 can be configured for generating at least a portion of the optical response signal indicative of the target gas composition within the ambient gas in the environment in response to the different illuminations. The signal processing circuitry 640 can be configured for processing the optical response signal at the different illuminations to determine a presence or other characteristic of the target gas composition within the ambient gas in the environment.

In an example, the signal processing circuitry 620 can include conductivity measurement circuitry electrically coupled to the functionalized region 610 to measure the conductivity of the functionalized region 610. The signal processing circuitry 620 can also include spectral response measurement circuitry for measuring the spectral response, and evaluation circuitry generating an indication of a concentration of the target gas composition of the ambient gas in the environment based on the measured conductivity and the measured spectral response.

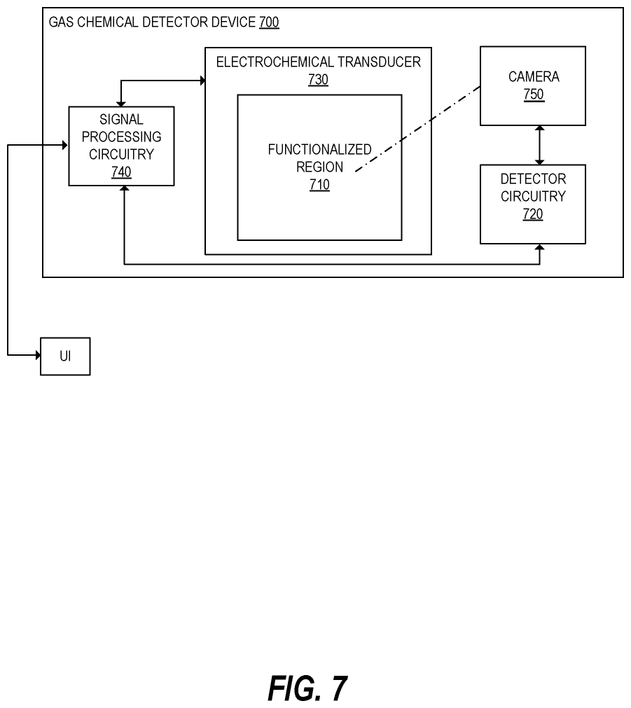

FIG. 7 is a block diagram of another example of a gas chemical detector device. A gas chemical detector device 700 can include or use an electrochemical transducer 730 including a functionalized region 710, detector circuitry 720, signal processing circuitry 740, and a camera 750. In an example, the camera 750 is arranged for imaging the functionalized region 710 such as to produce an image-readable characteristic indicative of the target gas composition within the ambient gas in the environment. Here, the signal processing circuitry 740 can include or use image-processing circuitry for processing an image of the functionalized region 710 such as to detect the image-readable characteristic. The signal processing circuitry 740 can include or use one or more circuits and/or algorithms, such as digital signal processing (DSP) circuits or algorithms, capable of analyzing the signal from the electrochemical transducer 730 and/or the processor in accordance with one or more parameters relevant to gas chemical detector device 700 to produce an output signal based on such analysis. For example, the signal processing circuitry 740 can include or use one or more algorithms capable of extracting data from the specimen image, such as data compression, filtering, edge detection, corner detection, blob detection, ridge detection, Hough transform, image segmentation, optical flow, genetic algorithms (GA), or other techniques for algorithmically analyzing the image. The one or more algorithms can be capable of digital image processing such as to extract relevant data from the specimen image to enable recognition of one or more relevant optical parameters.

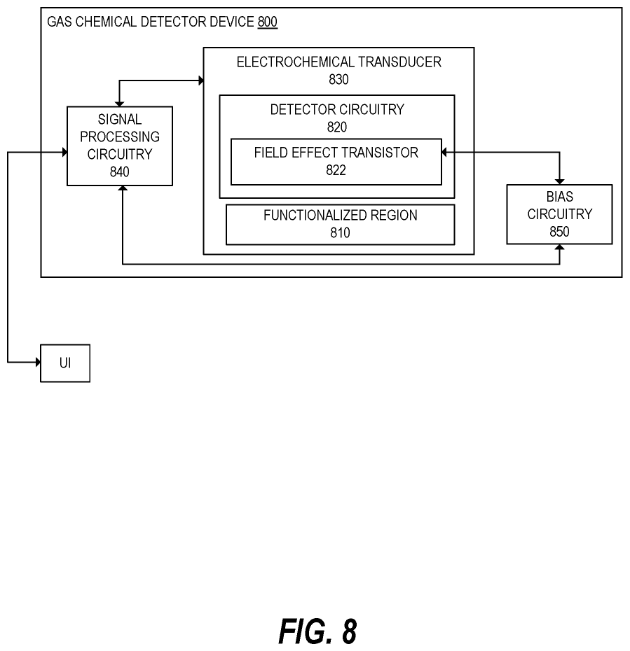

FIG. 8 is a block diagram of another example of a gas chemical detector device. A gas chemical detector device 800 can include or use an electrochemical transducer 830 including a functionalized region 810, detector circuitry 820, and signal processing circuitry 840. In an example, a field effect transistor (FET) 822 can be included in or can be coupled to the functionalized region 810. For example, the FET 822 can be arranged such as to use an electrical property of the functionalized region to transduce an electrical response signal. In an example, the functionalized region 810 can change an electrical property of the FET 822, e.g., an effective gate voltage of the FET 822, an effective channel resistance of the FET 822, an effective channel conductance of the FET 822, a transconductance of the FET 822, or at least one gate-body, gate-drain, or gate-source interface parameter of the FET 822.

Also, the gas chemical detector device 800 can include or use bias circuitry 850 coupled to the FET 822. The bias circuitry 850 can bias or pre-bias the FET 822. For example, the bias circuitry 850 can be configured to sweep the gate bias of FET 822. That is, in response to a gate bias sweep that sweeps a bias range or operating range of FET 822, the gas chemical detector device 800 can detect FET drain currents and their switching points during the sweep. The detection of the FET drain current switching points can provide a measurement of the threshold voltage of the FET 822. In other embodiments, the bias circuitry 850 can be configured to vary the gate voltage of FET 822. The bias circuitry 850 can include multiple stages that vary the bias of the FET 822.

The signal processing circuitry 840 can include a processing or conditioning circuit (e.g., an amplifier or a gain stage) for buffering, amplifying, and/or filtering the electrical response signal. Also, the signal processing circuitry 840 can include or use other electronic circuitry 850 for conditioning the electrical response signal. For example, other electronic circuitry 850 can include a pre-amplifier, filter circuits, a discriminator, an analog to digital converter, and a microprocessor or controller for configuring or providing one or more user interfaces. In an example, the gas chemical detector device 800 can provide an analog or a digital output signal.

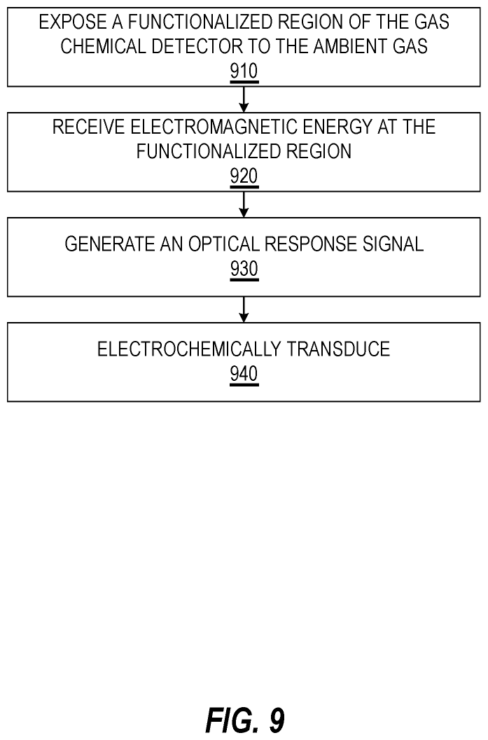

FIG. 9 is a flowchart that describes a method for detecting a target gas composition. In an example, at 910, the method can include exposing a functionalized region of the gas chemical detector to the ambient gas.

At 920, the method can include receiving electromagnetic energy at the functionalized region. For example, the method can include illuminating the functionalized region with the electromagnetic energy using at least one of a broadband or a tunable wavelength light source. For example, the illumination can be varied between a plurality of different illuminations. In an example, varying the illumination between a plurality of different illuminations comprises varying an illumination wavelength among individual ones of the plurality of different illuminations.

At 930, the method can include generating an optical response signal based on the optical property and indicative of the target gas composition within the ambient gas in the environment using the functionalized region. The optical response signal can be analyzed such as to determine a presence or other characteristic of the target gas composition within the ambient gas in the environment. This can include comparing or analyzing spectral response data from the optical response signal using at least one of a library template or trained model. In an example, the at least one of the library template or the trained model can be included such as to determine a presence or other characteristic of the target gas composition within the ambient gas in the environment for a Volatile Organic Compound (VOC) corresponding with one or more bacteria. In an example, generating at least a portion of the optical response signal can include generating spectral response data including at least one of absorption, reflection, fluorescence, elastic scattering, inelastic (Raman) scattering of the functionalized region, in response to an illumination.

At 940, the method can include electrochemically transducing an electrical property indicative of the target gas composition into an electrical response signal. For example, both the optical response signal and the electrical response signal can be processed, such as separately or together, such as concurrently or otherwise, to determine a presence or other characteristic of the target gas composition within the ambient gas in the environment. In an example, the electrical property can be electrochemically transduced into the electrical response signal using the same or a different functionalized region of the gas chemical detector. In an example, the functionalized region can change an electrical property of a field effect transistor (FET) in response to the functionalized region being exposed to ambient gas including the target gas composition. In an example, an electrical bias applied to the FET using a plurality of different electrical bias values.

Also, the method can include imaging the functionalized region to produce an image-readable characteristic indicative of the target gas composition within the ambient gas in the environment using the functionalized region. The method can include using the image-readable characteristic from the image-processing of the image of the functionalized region, generating at least a portion of the optical response signal indicative of the presence or other characteristic of the target gas composition within the ambient gas in the environment. For example, image-processing an image of the functionalized region can use colorimetry or spectral image analysis to detect the image-readable characteristic.

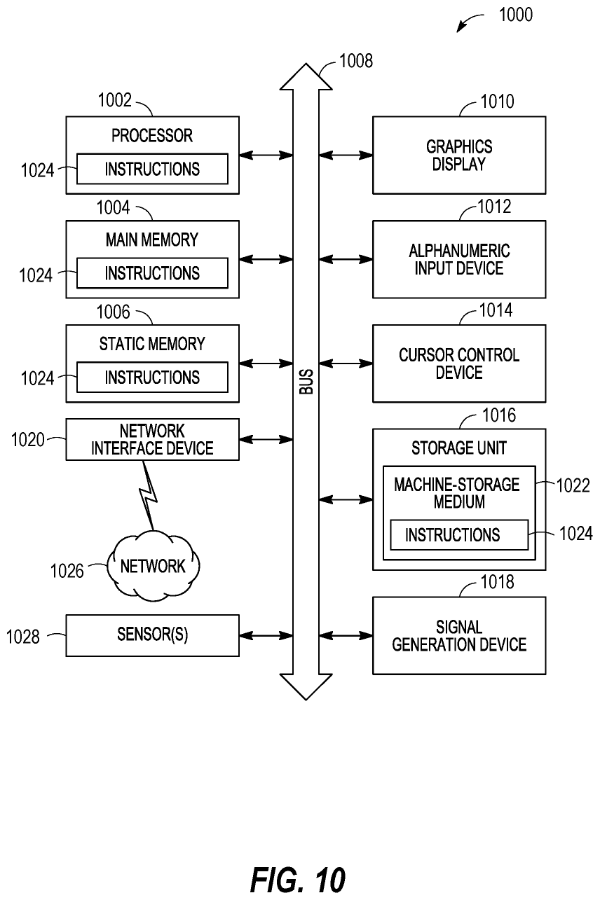

FIG. 10 is a block diagram illustrating components of a machine 1000, according to some example embodiments, able to read instructions 1024 from a machine-storage medium 1022 (e.g., a non-transitory machine-storage medium, a machine-storage medium, a computer-storage medium, or any suitable combination thereof) and perform any one or more of the methodologies discussed herein, in whole or in part. Specifically, FIG. 10 shows the machine 1000 in the example form of a computer system (e.g., a computer) within which the instructions 1024 (e.g., software, a program, an application, an applet, an app, or other executable code) for causing the machine 1000 to perform any one or more of the methodologies discussed herein can be executed, in whole or in part. For example, the instructions 1024 can be processor executable instructions that, when executed by a processor of the machine 1000, cause the machine 1000 to perform the operations outlined above.

In alternative embodiments, the machine 1000 operates as a standalone device or can be communicatively coupled (e.g., networked) to other machines. In a networked deployment, the machine 1000 can operate in the capacity of a server machine or a client machine in a server-client network environment, or as a peer machine in a distributed (e.g., peer-to-peer) network environment. The machine 1000 can be a server computer, a client computer, a personal computer (PC), a tablet computer, a laptop computer, a netbook, a cellular telephone, a smartphone, a set-top box (STB), a personal digital assistant (PDA), a web appliance, a network router, a network switch, a network bridge, or any machine capable of executing the instructions 1024, sequentially or otherwise, that specify actions to be taken by that machine. Further, while only a single machine is illustrated, the term “machine” shall also be taken to include any collection of machines that individually or jointly execute the instructions 1024 to perform all or part of any one or more of the methodologies discussed herein.

The machine 1000 includes a processor 1002 (e.g., a central processing unit (CPU), a graphics processing unit (GPU), a digital signal processor (DSP), an application specific integrated circuit (ASIC), a radio-frequency integrated circuit (RFIC), or any suitable combination thereof), a main memory 1004, and a static memory 1006, which are configured to communicate with each other via a bus 1008. The processor 1002 can contain microcircuits that are configurable, temporarily or permanently, by some or all of the instructions 1024 such that the processor 1002 is configurable to perform any one or more of the methodologies described herein, in whole or in part. For example, a set of one or more microcircuits of the processor 1002 can be configurable to execute one or more modules (e.g., software modules) described herein.

The machine 1000 can further include a graphics display 1010 (e.g., a plasma display panel (PDP), a light emitting diode (LED) display, a liquid crystal display (LCD), a projector, a cathode ray tube (CRT), or any other display capable of displaying graphics or video). The machine 1000 can also include an alphanumeric input device 1012 (e.g., a keyboard or keypad), a cursor control device 1014 (e.g., a mouse, a touchpad, a trackball, a joystick, a motion sensor, an eye tracking device, or other pointing instrument), a storage unit 1016, an audio generation device 1018 (e.g., a sound card, an amplifier, a speaker, a headphone jack, any suitable combination thereof, or any other suitable signal generation device), and a network interface device 1020.

The storage unit 1016 includes the machine-storage medium 1022 (e.g., a tangible and non-transitory machine-storage medium) on which are stored the instructions 1024, embodying any one or more of the methodologies or functions described herein. The instructions 1024 can also reside, completely or at least partially, within the main memory 1004, within the processor 1002 (e.g., within the processor's cache memory), or both, before or during execution thereof by the machine 1000. Accordingly, the main memory 1004 and the processor 1002 can be considered machine-storage media (e.g., tangible and non-transitory machine-storage media). The instructions 1024 can be transmitted or received over the network 1026 via the network interface device 1020. For example, the network interface device 1020 can communicate the instructions 1024 using any one or more transfer protocols (e.g., Hypertext Transfer Protocol (HTTP)).

In some example embodiments, the machine 1000 can be a portable computing device, such as a smart phone or tablet computer, and have one or more additional input components (e.g., sensors 1028 or gauges). Examples of the additional input components include an image input component (e.g., one or more cameras), an audio input component (e.g., a microphone), a direction input component (e.g., a compass), a location input component (e.g., a global positioning system (GPS) receiver), an orientation component (e.g., a gyroscope), a motion detection component (e.g., one or more accelerometers), an altitude detection component (e.g., an altimeter), and a gas detection component (e.g., a gas sensor). Inputs harvested by any one or more of these input components can be accessible and available for use by any of the modules described herein.

### Executable Instructions and Machine-Storage Medium

The various memories (i.e., 1004, 1006, and/or memory of the processor(s) 1002) and/or storage unit 1016 can store one or more sets of instructions and data structures (e.g., software) 1024 embodying or utilized by any one or more of the methodologies or functions described herein. These instructions, when executed by processor(s) 1002 cause various operations to implement the disclosed embodiments.

As used herein, the terms “machine-storage medium,” “device-storage medium,” “computer-storage medium” (referred to collectively as “machine-storage medium 1022”) mean the same thing and can be used interchangeably in this disclosure. The terms refer to a single or multiple storage devices and/or media (e.g., a centralized or distributed database, and/or associated caches and servers) that store executable instructions and/or data, as well as cloud-based storage systems or storage networks that include multiple storage apparatus or devices. The terms shall accordingly be taken to include, but not be limited to, solid-state memories, and optical and magnetic media, including memory internal or external to processors. Specific examples of machine-storage media, computer-storage media, and/or device-storage media 1022 include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms machine-storage medium or media, computer-storage medium or media, and device-storage medium or media 1022 specifically exclude carrier waves, modulated data signals, and other such media, at least some of which are covered under the term “signal medium” discussed below. In this context, the machine-storage medium is non-transitory.

### Signal Medium

The term “signal medium” or “transmission medium” shall be taken to include any form of modulated data signal, carrier wave, and so forth. The term “modulated data signal” means a signal that has one or more of its characteristics set or changed in such a matter as to encode information in the signal.

### Computer Readable Medium

The terms “machine-readable medium,” “computer-readable medium” and “device-readable medium” mean the same thing and can be used interchangeably in this disclosure. The terms are defined to include both machine-storage media and signal media. Thus, the terms include both storage devices/media and carrier waves/modulated data signals.

The above Detailed Description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A gas chemical detector device for detecting a presence or other characteristic of a target gas composition within ambient gas in an environment, the device comprising:
a functionalized region, configured to be exposed to the ambient gas, wherein the functionalized region is configured to include at least one of an electrical or optical property indicative of the target gas composition;
detector circuitry, configured for generating an optical response signal indicative of the target gas composition within the ambient gas in the environment using the functionalized region;
an electrochemical transducer for transducing an electrical property indicative of the target gas composition into an electrical response signal; and
signal processing circuitry, using both the optical response signal and the electrical response signal to determine a presence or other characteristic of the target gas composition within the ambient gas in the environment.

2. The device of claim 1, comprising a sensor including the functionalized region, the sensor including at least one of an electrochemical transducer, a photodetector, or a colorimetric imaging sensor.

3. The device of claim 2, wherein the sensor is located facing an opposing illuminator, and arranged to permit the ambient gas from the environment to enter a space between the sensor and the illuminator.

4. The device of claim 2, wherein:
the sensor includes an array of carbon nanotube field-effect transistors (CNFETs); and
individual ones of the CNFETs in the array of CNFETS correspond to different functionalized regions corresponding to different target gas compositions.

5. The device of claim 4, wherein the individual ones of the CNFETs include respective gate regions that are coated with different functionalized region material coatings to include at least one different electrical or optical property indicative of a particular target gas composition.

6. The device of claim 1, wherein the functionalized region includes at least one of an oligonucleotide, a metal coordination complex, a porphyrin, a self-assembled monolayer (SAM), a polymer, a pyrrole derivative, a phthalocyanine, or a nanomaterial decoration.

7. The device of claim 1, comprising:
a camera or other imager, arranged for imaging the functionalized region to produce an image-readable characteristic indicative of the target gas composition within the ambient gas in the environment using the functionalized region;
wherein the signal processing circuitry is configured for image-processing an image of the functionalized region to detect the image-readable characteristic and, using the image-readable characteristic from the image-processing of the image of the functionalized region, generating at least a portion of the optical response signal indicative of the presence or other characteristic of the target gas composition within the ambient gas in the environment.

8. The device of claim 1, comprising:
an illuminator, including at least one of a broadband or tunable wavelength light source, arranged to illuminate with electromagnetic energy the functionalized region exposed to the ambient gas;
wherein the detector circuitry is configured for generating at least a portion of the optical response signal indicative of the target gas composition within the ambient gas in the environment using the functionalized region, in response to the illuminating.

9. The device of claim 8, wherein:
the functionalized region is arranged to, in response to the illumination, provide optical response data including spectral response data representing a spectral characteristic of the functionalized region exposed to the ambient gas, the spectral characteristic including at least one of absorption, reflection, fluorescence, elastic scattering, inelastic (Raman) scattering indicative of the presence or other characteristic of the target gas composition within the ambient gas in the environment at the illumination.

10. The device of claim 8, comprising:
illumination controller circuitry, configured for varying the illumination between a plurality of different illuminations;
wherein the functionalized region is configured for generating at least a portion of the optical response signal indicative of the target gas composition within the ambient gas in the environment in response to the different illuminations; and
wherein the signal processing circuitry is configured for processing the optical response signal at the different illuminations to determine a presence or other characteristic of the target gas composition within the ambient gas in the environment.

11. The device of claim 1, wherein the electrochemical transducer includes or is coupled to the same or a different functionalized region of the device to use the electrical property of the functionalized region to transduce the electrical response signal.

12. The device of claim 1, wherein the detector circuitry includes a Field Effect Transistor (FET), including or coupled to the functionalized region, wherein the FET is configured for generating the optical response signal indicative of the target gas composition within the ambient gas in the environment.

13. The device of claim 12, comprising bias circuitry arranged for biasing the FET at a specified bias level, corresponding to the target gas composition, to determine a presence or other characteristic of the target gas composition within the ambient gas in the environment.

14. The device of claim 13, wherein the bias circuitry is configured to change an electrical bias applied to the FET, and an illumination controller operatively coupled to the illuminator is configured to change the illumination among a plurality of different illuminations.

15. The device of claim 12, wherein the functionalized region is configured to change an electrical property of the FET in response to the functionalized region being exposed to ambient gas including the target gas composition.

16. The device of claim 15, wherein the functionalized region is configured to change an electrical property of the FET including at least one of an effective gate voltage of the FET, an effective channel resistance of the FET, an effective channel conductance of the FET, a transconductance of the FET, or at least one gate-body, gate-drain, or gate-source interface parameter of the FET.

17. The device of claim 12, wherein the functionalized region is configured to vary a gate voltage of the FET as a function of the presence or other characteristic of the target gas composition within the ambient gas in the environment.

18. The device of claim 1, wherein the signal processing circuitry includes or is coupled to at least one of a library template or a trained model, wherein the signal processing circuitry is configured for processing using the optical response signal to determine a presence or other characteristic of the target gas composition within the ambient gas in the environment using the at least one of a library template or the trained model.

19. A method for detecting a target gas composition, within ambient gas in an environment, using a gas chemical detector, the method comprising:
exposing a functionalized region of the gas chemical detector to the ambient gas, wherein the functionalized region is configured to include an optical property indicative of the target gas composition;
receiving electromagnetic energy at the functionalized region;
generating an optical response signal based on the optical property and indicative of the target gas composition within the ambient gas in the environment using the functionalized region;
electrochemically transducing an electrical property indicative of the target gas composition into an electrical response signal; and
processing using both the optical response signal and the electrical response signal to determine a presence or other characteristic of the target gas composition within the ambient gas in the environment.

20. A gas chemical sensing device comprising:
an illumination source, arranged to provide an electromagnetic energy illumination;
a gas chemical detector, including one or more functionalized gas chemical detector regions arranged to receive the illumination from the illumination source and to be exposed to an ambient gas from an environment to be sensed, the one or more regions functionalized to modulate at least one of an electrical conductivity or a spectral optical response characteristic, the gas chemical detector including:
a functionalized semiconductor gas chemical detector first region, including a first functionalization material selected to modulate a conductivity of the first region, responsive to a concentration of a first specified gas composition; and
a functionalized semiconductor gas chemical detector second region, including a second functionalization material selected to provide optical response data including spectral response data representing a spectral characteristic of the functionalized region in response to being exposed to the ambient gas, the spectral characteristic including at least one of absorption, reflection, fluorescence, elastic scattering, inelastic (Raman) scattering indicative of a presence or other characteristic of a target gas composition within ambient gas in an environment at the illumination; and
signal processing circuitry, including conductivity measurement circuitry electrically coupled to the first region to measure the conductivity of the first region, and spectral response measurement circuitry using the second region to measure the spectral response characteristic of the second region, and evaluation circuitry generating an indication of a concentration of the target gas composition of the ambient gas in the environment based on the measured conductivity and the measured spectral response.
